(19) United States
(12) Patent Application Publication
(10) Pub. No.: US 2021/0052501 A1
(43) Pub. Date: Feb. 25, 2021

(54) METHOD AND APPARATUS

(71) Applicant: UNIVERSITY OF LIMERICK, Limerick (IE)

(72) Inventors: Ahmad B. ALBADARIN, Limerick (IE); Gavin WALKER, Limerick (IE); Jacek ZEGLINSKI, Limerick (IE)

(21) Appl. No.: 16/965,828

(22) PCT Filed: Jan. 24, 2019
(86) PCT No.: PCT/EP2019/051713

§ 371 Date: Jul. 29, 2020

(30) Foreign Application Priority Data
Jan. 29, 2018 (GB) 1801415.9

(51) Int. Cl. A61K 9/16; A61K 31/167; A61J 3/02

(52) U.S. Cl. CPC A61K 9/1688; A61J 3/02; A61K 9/1617; A61K 31/167

(57) ABSTRACT

A method of processing an active pharmaceutical ingredient, the method comprising passing a precursor composition comprising the active pharmaceutical ingredient and a solvent through a twin screw extruder wherein the twin screw extruder is heated at ambient pressure.

The present invention relates to a method of processing active pharmaceutical ingredients and to apparatus for use in such methods.

In particular the present invention relates to the use of twin screw extruders in the processing of active pharmaceutical ingredients (APIs).

Prior to their incorporation into a formulation for dosage, active pharmaceutical ingredients need to be provided in a ready to handle form which is highly purified and comprises minimum levels of solvent. Most APIs are desirably provided as dry materials in particulate form, as powders or granules. The size of these particles needs to be uniform and it is necessary for a synthetic method to provide particles of consistent size.

The synthesis of APIs typically involves a number of steps often including a crystallisation step as a purification means to provide the actual active ingredient. The isolated crystals may still contain high levels of solvent (typically more than 5 wt % and often much higher) and are not usually of a consistent size or indeed the desired size for inclusion in a pharmaceutical formulation.

Thus additional steps are usually needed after crystallisation to provide an API in a form ready for formulation. This further processing will usually involve a milling step and/or granulation step to provide particles of the desired size distribution and a drying step. In current methods the granulation and drying steps are carried out separately and usually involve batch processes.

For example particles may be granulated using high shear mixer granulators or twin screw extruders. The material is then dried using fluid bed dryers, vacuum drying, microwave drying, spray dryers, tray dryers, rotary core dryers, paddle dryers, tumble dryers or belt dryers.

However these drying processes take up considerable space and can be labour intensive. The product often requires further post-drying processing such as milling, screening or blending to provide homogeneity.

The present invention seeks to provide a method of processing active pharmaceutical ingredients which offers advantages over methods of the prior art.

According to a first aspect of the present invention there is provided a method of processing an active pharmaceutical ingredient, the method comprising passing a precursor composition comprising the active pharmaceutical ingredient and a solvent through a twin screw extruder wherein the twin screw extruder is heated at ambient pressure.

The present invention uses a twin screw extruder. This is a standard piece of equipment comprising a barrel and two screws which are located within the barrel. Rotation of the screws applies forces on to material fed into the barrel and delivers it along the barrel, typically from one end to the other. Depending on the nature of the apparatus the screws may be intermeshing or non-intermeshing. They may be configured to corotate or counter rotate. Twin screw extruders are very flexible and can be used to compress, mix, shear or shape a material delivered into the extruder.

The use of a heated twin screw extruder to process polymeric materials is well known. However for APIs the extruder is not usually heated. Thus in methods of the prior art extrusion has been used for granulation but has then been followed by a separate drying step.

The present invention advantageously involves a combined heating and drying step. Thus the invention suitably provides in a single step an API as a dry particulate material which can be directly incorporated into formulations without the need for any additional processing steps.

The method of the present invention involves passing a precursor composition comprising an API and a solvent through a twin screw extruder.

By passing through a twin screw extruder, we mean that the composition is delivered into an inlet of the extruder, is subjected to processing within the extruder, and then is ejected from an outlet of the extruder.

Depending on the configuration of the screws within the barrel of the extruder, the inlet and outlet may be positioned at any part of the extruder. In preferred embodiments an inlet is provided at one end of the barrel, an outlet is provided at the other end of the barrel and the screws are configured to carry the composition along the length of the barrel.

The skilled person will appreciate that the material discharged from the extruder at the end of the process (“the treated composition”) will be different in form and nature to “the precursor composition” fed into the extruder.

The precursor composition comprises an active pharmaceutical ingredient and a solvent. In some embodiments the precursor composition may comprise a mixture of two or more active pharmaceutical ingredients and/or two or more solvents.

The precursor composition may be provided in any suitable form. Preferably it is provided as a solid composition. Suitably it is provided as a “wet” solid. The precursor composition may be the solid recovered from a recrystallisation step, for example a wet filter cake.

The precursor composition comprises one or more APIs and one or more solvents. The precursor composition may comprise one or more further components, for example one or more pharmaceutically acceptable excipients. However in preferred embodiments the APIs and solvents together provide at least 90 wt % of the precursor composition, preferably at least 95 wt %, suitably at least 99 wt %, for example at least 99.9 wt % or at least 99.99 wt %.

The solvent(s) are suitably present in the precursor composition in an amount of at least 1 wt %, preferably at least 2.5 wt %, suitably at least 5 wt %. In some embodiments the solvent(s) are present in an amount of at least 8 wt %, for example at least 10 wt %, at least 12 wt % or at least 15 wt %. In some embodiments the solvent(s) may be present in an mount of more than 20 wt %.

Suitably the precursor composition comprises up to 60 wt % of one or more solvent(s), suitably up to 50 wt %, preferably up to 40 wt %, for example up to 35 wt %.

In some embodiments the precursor composition comprises from 10 to 40 wt % of one or more solvents, for example 10 to 20 wt %.

In some embodiments the precursor composition may comprise up to 50 wt % of one or more solvents.

The present invention may be used in the processing of any suitable API.

Suitable APIs typically exist in solid form under ambient conditions.

Preferably the API is not air sensitive.

Preferably the API is not moisture sensitive.

Suitable APIs for use herein include paracetamol, ibuprofen, aspirin, felodipine, piracetam, irbesartan, hydrochlorothiazide, caffeine, amoxiciline, griseofulvin, metformin, chlorpheniramine maleate, simvastatin, etoricoxib, eprosartan, levofloxacin and sevelamer.

Suitable API forms for use herein include pharmaceutical salts with cationic counterions including sodium, calcium, potassium, magnesium, lysine, etc., and anionic counterions including chloride, bromide, sulfate, acetate, etc.

Suitable API forms for use herein include hydrates.

Any suitable solvent may be included in the precursor composition. Preferred solvents are pharmaceutically acceptable solvents. Suitable solvents for use herein include aliphatic or cycloaliphatic hydrocarbons, aromatic hydrocarbons, halogenated aliphatic hydrocarbons, halogenated aromatic hydrocarbons, alcohols, ketones, ethers, esters, nitrated aromatic compounds, amines, amides and organic acids. Suitable solvents for use herein are described, for example, in U.S. Pat. No. 4,535,151 A.

Suitable solvents include water, ethanol, isopropanol, glycerol, ethyl acetate, hexane, cyclohexane, acetone, methanol, chloroform, toluene, dioxane, acetic acid and propylene glycol.

The precursor composition comprising the API(s) and solvent(s) is passed through a twin screw extruder wherein the twin screw extruder is heated at ambient pressure.

By heated at ambient pressure we mean that a vacuum is not applied to the barrel of the extruder and that pressurised gas is not pumped into the extruder. Suitably no gas is fed into the extruder.

The twin screw extruder may be heated by any suitable means. Preferably heating elements are provided around the barrel of the extruder and/or internally within the screws.

In some preferred embodiments the extruder barrel is fitted with three to nine longitudinally spaced-apart electric heating elements, each of which is independently controlled with separate thermostats attached to the barrel in the vicinity of the element which it controls. The first heating element is located near the feed inlet of the extruder, and the last near the extrusion orifice. The elements provide heat input for drying/extrusion of excipients and polymeric material at a constant, preselected temperature.

In some embodiments only a portion of the extruder is heated.

In preferred embodiments the extruder is heated along its length.

In some embodiments the temperature to which the extruder barrel is heated may vary along its length, for example the temperature of the material may increase progressively as it passes along the barrel.

Suitably at least a portion of the extruder is heated to a temperature above the boiling point of the solvent(s) present in the precursor composition.

Preferably the portion of the extruder is heated to a temperature below the melting point of the API.

In some preferred embodiments the extruder is heated to a temperature of at least 40° C., suitably at least 50° C. In some embodiments the extruder may be heated for a temperature of more than 60° C., for example more than 70° C. or more than 80° C.

Suitably the extruder is provided with one or more vent holes along its length to allow gas to escape. Thus solvent which evaporates is not retained within the extruder. This improves the drying efficiency of the system.

Suitably the extruder is long to ensure ample time for material to be processed to a desired size and dried.

Suitably the ratio of the length to the diameter of the extruder barrel is at least 10:1, preferably at least 20:1, suitably at least 25:1. In preferred embodiments the ratio of length to diameter is at least 30:1. It may be at least 35:1.

The ratio of length to diameter of the extruder barrel may be up to 100:1, suitably up to 80:1, for example up to 60:1 or up to 50:1.

In one preferred embodiment the ratio of length to diameter of the barrel of the extruder used to carry out the method of the present invention is about 40:1.

However the ratio of the length to diameter may vary depending on the particular conditions.

Advantageously the present invention provides a product which is highly homogeneous and has low solvent content.

Suitably the treated composition has a solvent content of less than 5 wt %, preferably less than 4 wt %, suitably less than 3 wt %, preferably less than 2 wt %.

In some embodiments the treated composition may have a solvent content of less than 1 wt %, preferably less than 0.5 wt %, for example less than 0.1 wt %.

The present invention suitably provides a treated composition having a high degree of homogeneity.

Suitably the particles of API have a substantial uniform size and a narrow particle size distribution. Preferably at least 80% of the particles have a diameter within 20% of the mean diameter. More preferably at least 90% of the particles have a diameter within 10% of the mean diameter.

The absolute size of the particles obtained by the present invention will depend on the API and the specific requirements of a particular formulation. The invention can be adapted to make particles of many different sizes. Typical particle sizes are from 50 to 1000 microns, suitably from 250 to 1000 microns.

Particle size may suitably be measured by Microtrac and Mastersizer laser diffraction or using scanning electron microscopy and/or optical microscopy.

Preferably the method of the present invention involves a continuous process. This offers a number of advantages over the batch processes of the prior art.

According to a second aspect of the present invention there is provided a method of preparing a formulated pharmaceutical product, the method comprising:
(a) providing an active pharmaceutical ingredient using the method of the first aspect; and
(b) admixing the treated composition obtained in step (a) with one or more further components.

Suitably there are no additional steps between step (a) and step (b).

Preferred features of step (a) are as defined in relation to the first aspect.

In step (b) the treated composition is admixed with one or more components.

The treated composition is suitably ejected from the extruder and admixed directly with one or more further components.

The one or more further components may be suitably selected from fillers, binders, colourings, flavourings, flow enhancers, taste maskers, lubricants, sufactants and other pharmaceutically acceptable excipients.

Following step (b) the mixture may be processed to form a tablet or used to fill a capsule.

This present invention can in a single step provide an API having the required moisture/solvent content and particle size distribution. The present invention is very simple compared with currently available continuous drying methods for example, spray drying. The invention does not involve complex expensive complicated equipment, the use of a vacuum or a preheated gas as a drying medium. It is also readily scaleable and thus advantageous for use in large scale industrial settings.

This invention does not cause agglomeration issues relating to dried APIs that can occur in other method and produces uniform particles having a narrow particle size distribution.

The product is obtained in high yield compared to currently used driers, due to the relatively low internal surface of the twin screw extruder and thus reduced deposition of the dried material inside the drying chamber.

The risks associated with handling toxic and/or explosive materials is minimised as a relatively small quantity of API will reside in the extruder barrel at any time in this continuous process, compared to the use of a large scale batch process.

The one-step method involves decreased material handling and reduced energy costs, compared to two stage processes of the prior art.

In prior wet granulation processes followed by a separate drying step agglomeration can occur during conveying and collection and there may be a change in the morphology and/or the porosity of the granules. This is much less likely to occur using the method of the present invention.

The invention also involves the integration of fewer operation units and thus reduces the processing cost and time.

According to a third aspect of the present invention there is provided apparatus for processing an active pharmaceutical ingredient, the apparatus comprising:
a barrel
two screws located within the barrel,
means for inputting material into the barrel,
exit means for collected material from the barrel,
one or more heating elements arranged to heat material inside the barrel,
one or more vents along the length of the barrel.

The apparatus of the third aspect is suitably a twin screw extruder. Preferred features of the third aspect are as defined in relation to the first and second aspects.

According to a fourth aspect of the present invention there is provided the use of a heated twin screw extruder to concurrently dry and granulate an active pharmaceutical ingredient.

The invention will now be further described with reference to the following non-limiting examples.

### EXAMPLE 1

A powder mixture of API/excipients blend was continuously fed into a twin screw granulator. After reaching a steady state of powder input/output, a binder feeder was connected to the barrel and the granulation process started. Then, heating elements were fitted to the barrel (with vents to allow vapour to escape) and the temperature of the heated zones was set at 50 degC. The granulated/dried particles were collected and the moisture content and particle size distribution (PSD) measured. The granules produced had a more uniformed PSD and a relatively low moisture content in comparison to the conventional twin screw granulation process.

### EXAMPLE 2

A mixture of paracetamol/ethanol (wet cake), with the solvent content of 28.3 wt % was continuously fed into a twin screw extruder (TSE) at three different flow rates: 28.9 g/h (A), 10.3 g/h (B), and 1.7 g/h (C). In preferred embodiments the extruder barrel is fitted with three to nine longitudinally spaced-apart electric heating elements, each of which is independently controlled with separate thermostats attached to the barrel in the vicinity of the element which it controls. The first heating element is located near the feed inlet of the extruder, and the last near the extrusion orifice. The TSE screw speed was set at 10 rpm and conveying elements were used in screw configuration. Heating elements were fitted to the barrel (with vents to allow vapour to escape) and the temperature of the heated zones was set at 83° C. The granulated/dried particles were collected and the moisture content and particle size distribution (PSD) measured.

Our results show that at those experimental conditions, the drying efficiency in the twin screw extruder of the model API material (paracetamol/ethanol “wet cake” (W)) was between 98.8% and 100%, thus the residual moisture was between 0% and 1.2%, depending on the specific conditions. Also, by changing processing conditions (particularly the flow rate) it was possible to tailor the distribution of particle sizes in order to avoid generating both fines and oversized particles.

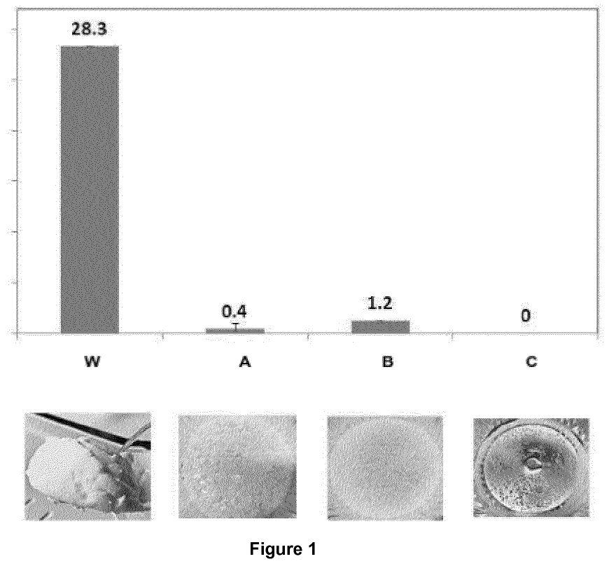

FIG. 1 shows the moisture content for flow rates A, B and C compared with the initial wet cake W and includes photographs of the product in each case.

1. A method of processing an active pharmaceutical ingredient, the method comprising passing a precursor composition comprising the active pharmaceutical ingredient and a solvent through a twin screw extruder wherein the twin screw extruder is heated at ambient pressure.

2. A method of preparing a formulated pharmaceutical product, the method comprising:
(a) providing an active pharmaceutical ingredient using the method of claim 1; and
(b) admixing the treated composition obtained in step (a) with one or more further components.

3. A method according to claim 1 or claim 2 wherein a barrel of the twin screw extruder includes one or more vents along its length.

4. A method according to claim 1 any preceding claim wherein the active pharmaceutical ingredient is selected from paracetamol, ibuprofen, aspirin, felodipine, piracetam, irbesartan, hydrochlorothiazide, caffeine, amoxiciline, griseofulvin, metformin, chlorpheniramine maleate, simvastatin, etoricoxib, eprosartan, levofloxacin and sevelamer.

5. A method according to claim 1 any preceding claim wherein the solvent is selected from water, ethanol, isopropanol, glycerol, ethyl acetate, hexane, cyclohexane, acetone, methanol and propylene glycol.

6. A method according to claim 1 any preceding claim wherein the precursor composition comprises at least 5 wt % solvent.

7. A method according to claim 1 any preceding claim which provides a treated composition comprising less than 1 wt % solvent.

8. Apparatus for processing an active pharmaceutical ingredient, the apparatus comprising:
a barrel,
two screws located within the barrel,
means for inputting material into the barrel,
exit means for collected material from the barrel,
one or more heating elements arranged to heat material inside the barrel, and
one or more vents along a length of the barrel.

9. (canceled)

10. A method according to claim 2, wherein a barrel of the twin screw extruder includes one or more vents along its length.

11. A method according to claim 2, wherein the active pharmaceutical ingredient is selected from paracetamol, ibuprofen, aspirin, felodipine, piracetam, irbesartan, hydrochlorothiazide, caffeine, amoxiciline, griseofulvin, metformin, chlorpheniramine maleate, simvastatin, etoricoxib, eprosartan, levofloxacin and sevelamer.

12. A method according to claim 2, wherein the solvent is selected from water, ethanol, isopropanol, glycerol, ethyl acetate, hexane, cyclohexane, acetone, methanol and propylene glycol.

13. A method according to claim 2, wherein the precursor composition comprises at least 5 wt % solvent.

14. A method according to claim 2, which provides a treated composition comprising less than 1 wt % solvent.
